(19) United States
(12) Patent Application Publication
(10) Pub. No.: US 2025/0359768 A1
(43) Pub. Date: Nov. 27, 2025

(54) DEVICE AND METHOD FOR AUTOMATIC AND REPETITIVE MEASUREMENT OF BLOOD PRESSURE IN SMART BLOOD PRESSURE MONITORS

(71) Applicant: NC MEDICAL CO.LTD, Yeongwol-gun (KR)

(72) Inventors: Chun Gwang BYUN, Seoul (KR); Ji Hyun BYUN, Seoul (KR)

(21) Appl. No.: 18/750,499

(22) Filed: Jun. 21, 2024

(30) Foreign Application Priority Data
May 27, 2024 (KR) 10-2024-0068739

(51) Int. Cl. A61B 5/021; A61B 5/00

(52) U.S. Cl. CPC A61B 5/02133; A61B 5/681; A61B 5/7264; A61B 5/7271; A61B 5/742; A61B 2560/045; A61B 2560/0462; A61B 2562/0247

(57) ABSTRACT

This invention, which offers the advantage of an automatic repetitive blood pressure measurement that minimizes discomfort for the subject while continuously measuring blood pressure regardless of the subject's movement, thereby enhancing the reliability of the measurement results, which leads to the development of an automatic repetitive blood pressure measurement device and method for a smart blood pressure monitor, relates to blood pressure measurement technology, involving periodically repeating the inflation and deflation of a cuff a set number of times upon receiving a single operation signal input. The method includes the steps of detecting arterial wall vibrations when the cuff pressure is between the systolic and diastolic pressures of the artery during cuff inflation and deflation, recognizing the point where maximum vibration is detected as the systolic blood pressure based on the detection results, recognizing the diastolic blood pressure if the vibration decrease exceeds a threshold, and providing measurement results sequentially, including the recognized systolic and diastolic blood pressure values during the repeated inflation and deflation of the cuff a set number of times.

### CROSS-REFERENCE TO RELATED APPLICATION(S)

This application claims priority to Korean Patent Application No. 10-2024-0068739 filed in the Korean Intellectual Property Office on May 27, 2024, the disclosure of which is incorporated by reference herein in its entirety.

### TECHNICAL FIELD

This invention relates to blood pressure measurement technology.

### BACKGROUND OF THE INVENTION

In the medical field, methods for measuring a patient's blood pressure are critical. Identifying whether a patient has hypertension or hypotension is essential for managing their condition, and various methods have been developed to measure blood pressure. The most basic way is to wrap a cuff around the subject's arm and exert pressure with the cuff to stop blood flow. Then, as the pressure applied by the cuff gradually decreases, the Korotkoff sounds are detected. The point at which the Korotkoff sounds begin is measured as the systolic blood pressure, and the point at which the Korotkoff sounds cease is measured as the diastolic blood pressure. Additionally, the oscillometric method, which also uses a cuff wrapped around the upper arm, measures blood pressure by detecting oscillations in the cuff pressure caused by blood flow. These oscillations (oscillometric waveforms) are then used in statistical and empirical regression equations to determine systolic and diastolic blood pressure.

Using a blood pressure cuff to apply pressure to the arm and measuring blood pressure through Korotkoff sounds provides high accuracy but has limitations because it requires a person with professional knowledge to measure it directly and it only captures blood pressure at a single moment, failing to show real-time changes in the subject's blood pressure. Moreover, if higher reliability is desired after checking the result of a single measurement or if unexpected results are obtained, the inconvenience of operating the blood pressure measuring device and performing repeated measurements arises. Additionally, phenomena such as white-coat uncontrolled hypertension or masked hypertension, where blood pressure temporarily rises in a medical setting, occur frequently, interfering with accurate blood pressure measurement. Therefore, rather than limiting blood pressure measurement to a single instance, performing multiple measurements can yield more accurate and reliable results, for example, by reducing individual measurement errors caused by temporary stress or environmental changes. Additionally, through repeated blood pressure measurements, the consistency and reliability of the measuring device can be verified, thereby enhancing the reliability of the blood pressure measurement results.

### SUMMARY

This invention has been derived from the same technical background, aiming to provide an automatic repetitive blood pressure measurement device and method for a smart blood pressure monitor that can continuously and automatically measure blood pressure regardless of the subject's movement while minimizing the subject's discomfort, thereby improving the reliability of the measurement results.

The present invention for achieving the above object includes the following configuration. The automatic repetitive blood pressure measurement method of the smart blood pressure monitor according to an embodiment of the present invention is performed in a computing device having one or more processors, and memory storing one or more programs executed by the one or more processors. The method periodically repeats the inflation and deflation of the cuff a set number of times upon receiving a single operation signal input, which includes a step of detecting the vibration of the arterial wall when the cuff pressure is between the systolic and diastolic pressures of the artery during the inflation and deflation of the cuff, identifying the point where the maximum vibration is detected as the systolic blood pressure, recognizing the point where the vibration reduction exceeds the threshold value as the diastolic blood pressure, and providing the measurement results sequentially, including the recognized systolic and diastolic blood pressure values during the set number of repetitions of the cuff inflation and deflation.

Meanwhile, an embodiment of an automatic repetitive blood pressure measurement of a smart blood pressure monitor includes one or more processors and a computer device having memory storing one or more programs executed by one or more processors. This device periodically repeats the inflation and deflation of the cuff a set number of times upon receiving a single operation signal input, which includes a detection unit that detects arterial wall vibrations when the cuff pressure is between the systolic and diastolic pressures of the artery during the inflation and deflation of the cuff, a blood pressure recognition unit that recognizes the point where the maximum vibration is detected as the systolic blood pressure and the point where the vibration reduction exceeds the threshold value as the diastolic blood pressure, and an Information provision unit that provides the measurement results sequentially, including the recognized systolic and diastolic blood pressure values during the set number of repetitions of the cuff inflation and deflation.

According to one embodiment of the present invention, while minimizing the subject's discomfort, it is possible to continuously measure blood pressure regardless of the subject's movement and improve the reliability of the measurement results, thereby providing an automatic repetitive blood pressure measurement of a smart blood pressure monitor and a method thereof.

### BRIEF DESCRIPTION OF THE DRAWINGS

FIG. 1 is a block diagram of an automatic repetitive blood pressure measurement of a smart blood pressure monitor according to an embodiment of the present invention.

FIG. 2 is an example of an external view of a wrist-worn automatic repetitive blood pressure measurement of a smart blood pressure monitor according to an embodiment.

FIGS. 3A and 3B are example diagrams explaining the form in which measurement results are provided from an Information Provision Unit according to an embodiment.

FIG. 4 is a flowchart explaining the method of automatic repetitive blood pressure measurement of a smart blood pressure monitor according to an embodiment of the present invention.

### DETAILED DESCRIPTION

The technical terms used in this invention are employed solely to describe specific embodiments and are not intended to limit the invention. Additionally, unless otherwise defined, the technical terms used in this invention should be interpreted as generally understood by those skilled in the art to which this invention pertains and should not be interpreted in an overly broad or overly narrow sense.

Referring to the attached illustrations, the preferred embodiments of the present invention will now be described in detail.

The automatic repetitive blood pressure measuring device, according to the embodiments of the present invention, can be implemented by at least one computer device, and the method for automatic repetitive blood pressure measurement of a smart blood pressure monitor, according to the embodiments of the present invention can be performed through at least one computer device included in the automatic repetitive blood pressure measurement of a smart blood pressure monitor. In this case, the computer device can have a computer program installed and run according to an embodiment of the present invention, and the computer device can perform the method for automatic repetitive blood pressure measurement of a smart blood pressure monitor according to the embodiments of the present invention under the control of the executed computer program.

The aforementioned computer program can be stored in a computer-readable recording medium to be executed on a computer in combination with the computer device to perform the method for automatic repetitive blood pressure measurement of a smart blood pressure monitor.

The automatic repetitive blood pressure measurement of a smart blood pressure monitor, according to one embodiment, continuously measures blood pressure (three times) with a single operation for one blood pressure measurement and displays all the measured blood pressure information for each cycle, which optimizes the blood pressure measurement method for existing users, enhances measurement convenience, and provides a bracelet-type three-sequence blood pressure monitor that can offer blood pressure information that the user demands or trusts. With just one operation, it automatically measures blood pressure repeatedly as many times as set, provides multiple blood pressure measurement results at once, and uses the accumulated measurement results to improve the accuracy of blood pressure measurement.

In other words, automatically repeating the blood pressure measurement several times with just one measurement operation eliminates the hassle of having to repeat multiple measurements manually. With a simple single operation, it can reduce individual measurement errors that may occur due to temporary stress or environmental changes and improve the reliability of the measurement results by confirming the consistency and reliability of the measuring device, which helps to monitor and maintain cardiovascular health more effectively.

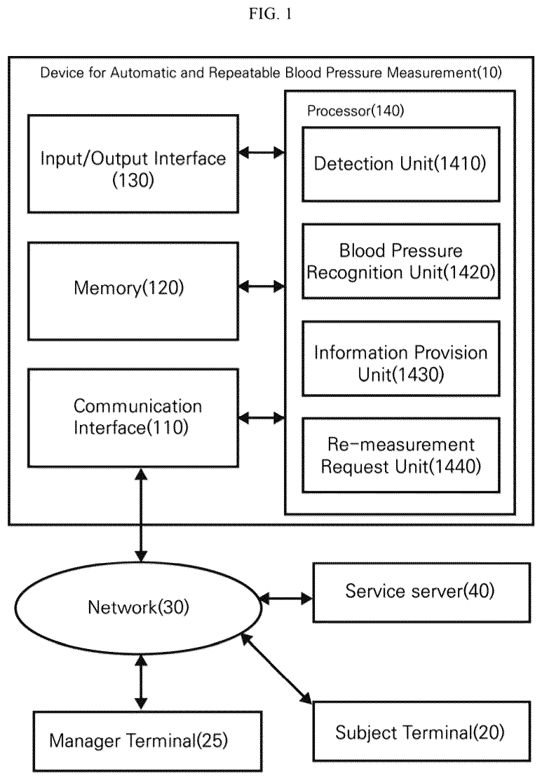

FIG. 1 is a block diagram of a automatic repetitive blood pressure measurement of a smart blood pressure monitor according to an embodiment of the present invention.

As shown in FIG. 1, the automatic repetitive blood pressure measurement of a smart blood pressure monitor (10), according to one embodiment, specifically includes a communication interface (110), memory (120), input/output interface (130), and a processor (140).

The communication interface (110) can provide the function for the automatic repetitive blood pressure measurement of a smart blood pressure monitor (10) to communicate with other devices through the network. For example, requests, commands, data, and files generated by the processor (140) of the automatic repetitive blood pressure measurement device (10) according to the program code stored in a storage device like memory (120) can be sent to at least one or more subject terminals (20), manager terminals (25), or service servers (40) through the network (30) under the control of the communication interface (110).

Conversely, signals, commands, data, files, etc., from other devices can be received through the communication interface (110) of the automatic repetitive blood pressure measurement device (10) via a network. The signals, commands, and data received through the communication interface (110) can be transmitted to the processor (140) or memory (120), and files, etc., can be stored in a storage medium (the aforementioned permanent storage device) that may be further included in the automatic repetitive blood pressure measurement device (10). The network (30) can include one or more networks such as PAN (personal area network), LAN (local area network), CAN (campus area network), MAN (metropolitan area network), WAN (wide area network), BBN (broadband network), the internet, and others. Additionally, the network can include any one or more of the following network topologies: bus network, star network, ring network, mesh network, star-bus network, tree or hierarchical network, etc., but is not limited to these.

The subject terminal (20) and manager terminal (25) can be applied to various devices such as smartphones, portable terminals, mobile terminals, foldable terminals, personal digital assistants (PDAs), portable multimedia players (PMPs), telematics terminals, navigation devices, personal computers, notebook computers, slate PCs, tablet PCs, ultrabooks, wearable devices (e.g., smartwatches, smart glasses, head-mounted displays (HMDs)), Wibro devices, IPTV (Internet Protocol Television) devices, smart TVs, digital broadcasting terminals, AVN (Audio Video Navigation) devices, A/V (Audio/Video) systems, flexible terminals, and digital signage devices.

In one embodiment, the subject terminal (20) can be interpreted to include the terminal device possessed by the blood pressure subject who wishes to measure their blood pressure using the automatic repetitive blood pressure measurement of a smart blood pressure monitor (10). The subject terminal (20) can receive the blood pressure measurement results from the automatic repetitive blood pressure measurement of a smart blood pressure monitor (10) and additionally receive analysis results information regarding the measured blood pressure results.

In one embodiment, the manager terminal (25) can be interpreted to include terminal devices possessed by medical personnel and others who perform medical activities using the blood pressure measurement results obtained from the automatic repetitive blood pressure measurement of a smart blood pressure monitor (10). However, the managers who possess the manager terminal (25) are not limited to medical personnel, they can also include blood pressure data managers from various professions who use the blood pressure measurement results to provide services.

The service server (40) can be implemented in the form of a web server, database server, proxy server, etc. The service server (40) may have one or more types of software installed that enable network load balancing or allow the service server (40) to operate on the internet or other networks, thereby implementing a computerized system. Additionally, the network can be an HTTP network, a private line, an intranet, or any other type of network. Furthermore, the connection between the automatic repetitive blood pressure measurement of a smart blood pressure monitor (10), the subject terminal (20), and the manager terminal (25) can be through a secure network to prevent data attacks by hackers or other third parties. The service server (40) can also include multiple database servers, which can be implemented separately from the service server (40) through any type of network connection, including a distributed database server architecture.

In one embodiment, the service server (40) receives blood pressure measurement results from the automatic repetitive blood pressure measurement of a smart blood pressure monitor (10), matches them with subject information, and stores them. It can provide various forms of information and services using the blood pressure measurement results to the subject terminal (20) and the manager terminal (25).

The memory (120) is a computer-readable recording medium and may include RAM (random access memory), ROM (read-only memory), and non-volatile mass storage devices (such as disk drives and servers). Here, non-volatile mass storage devices such as ROM and disk drives are separate permanent storage devices distinct from the memory (120) and may be included in the automatic repetitive blood pressure measurement of a smart blood pressure monitor (10). The memory (120) can also store an operating system and at least one program code. These software components can be loaded into the memory (120) from a computer-readable recording medium that is separate from the memory (120). Such computer-readable recording media can include floppy drives, disks, tapes, DVD/CD-ROM drives, memory cards, SSDs, USB drives, and other computer-readable recording media.

In another embodiment, software components can be loaded into the memory (120) through the communication interface (110) instead of a computer-readable recording medium. For example, the software components can be loaded into the memory (120) of the automatic repetitive blood pressure measurement of a smart blood pressure monitor (10) based on computer programs installed by files received through the network.

The processor (140) can be configured to process commands of a computer program by performing basic arithmetic, logic, and input/output operations. Commands can be provided to the processor (140) by the memory (120) or the communication interface (110). For example, the processor (140) can be configured to execute commands received according to program code stored in a storage device like the memory (120).

As shown in FIG. 1, in one embodiment, the processor (140) of the automatic repetitive blood pressure measurement of a smart blood pressure monitor (10) includes more specific components: a detection unit (1410), a blood pressure recognition unit (1420), an information provision unit (1430), and a re-measurement request unit (1440).

The detection unit (1410) first receives information on the number of repetitions for measuring blood pressure from medical personnel or a blood pressure data manager upon receiving a single operation signal. The repetition measurement cycle information can be input through the input/output interface (130) provided in the automatic repetitive blood pressure measurement of a smart blood pressure monitor (10). However, it is not limited to this and can also be implemented to receive settings from the manager terminal (25) via the network (30).

For example, the manager terminal (25) can be implemented to perform control and management functions for at least one automatic repetitive blood pressure measurement of a smart blood pressure monitor (10). The manager terminal (25) can receive repetition measurement cycle information for each automatic repetitive blood pressure measurement of a smart blood pressure monitor (10) individually.

Also, the detection unit (1410) is implemented to perform the inflation and deflation of the cuff for the preset number of repetitions when a single operation signal is input. The cuff contains gas inside, which can be variable. That is, the internal pressure can change according to the amount of gas.

The detection unit (1410) periodically repeats the inflation and deflation of the cuff for the set number of repetitions upon receiving a single operation signal. The inflation of the cuff compresses the artery, causing the cuff pressure to become higher than the arterial pressure, temporarily blocking the arterial flow.

Then, when the cuff deflates and the cuff pressure becomes lower than the systolic blood pressure of the artery, blood flows through the artery. During this process, the arterial wall vibrates (pulse wave) as the blood flow is temporarily blocked or allowed to flow. The detection unit (1410) can detect these vibrations in the arterial wall. Specifically, the Detection unit (1410) can include a pressure sensor located inside the cuff to detect changes in the cuff's internal pressure in real time. The detected vibration signals can then be analyzed using digital signal processing techniques to digitize the signals.

Specifically, the detected electrical signals are amplified using an amplifier, and filtering is performed using a low-pass filter and another low-pass filter to remove noise and unnecessary signals.

In other words, the detection unit (1410) can detect the vibrations of the arterial wall when the cuff pressure is between the systolic and diastolic pressures of the artery, either when the blood flow is temporarily blocked and then resumes or when the blood flow is temporarily allowed and then blocked.

In one embodiment, the detection unit (1410) is implemented as a high-speed sensor that can quickly detect pressure changes and process data in real-time, thereby improving the efficiency of the measurement process and reducing measurement time. For example, it can be implemented as a piezoresistive pressure sensor that uses the property of changing resistance when pressure is applied, a capacitive pressure sensor that measures pressure by detecting changes in capacitance caused by the varying distance between two electrodes, a piezoelectric pressure sensor that uses the principle of generating voltage when a material is subjected to pressure, or a MEMS (Micro-Electro-Mechanical Systems) sensor. However, it is not limited to these implementations.

The blood pressure recognition unit (1420) recognizes the systolic blood pressure at the point where the maximum vibration is detected according to the arterial wall vibration detection results from the detection unit (1410), and it recognizes the diastolic blood pressure if the amount of vibration decrease exceeds a certain threshold.

Additionally, the blood pressure recognition unit (1420) can derive accurate systolic and diastolic blood pressure by recognizing the vibration signal patterns of the arterial wall.

The blood pressure recognition unit (1420) uses a special algorithm to interpret the vibration patterns and calculate systolic and diastolic blood pressure. This special algorithm can accurately calculate blood pressure values by analyzing the magnitude and frequency of the vibrations.

In one embodiment, the blood pressure recognition unit (1420) can further detect the pulse pressure value while repeating the inflation and deflation of the cuff the set number of times upon receiving a single operation signal input.

Pulse pressure (PP) is the difference between systolic blood pressure (SBP) and diastolic blood pressure (DBP), which can reflect the elasticity (compliance) of the arteries. In other words, it can help determine the risk of cardiovascular diseases by indicating whether the arteries have stiffened or if arteriosclerosis has progressed.

Additionally, it can indicate whether there is an increased risk of conditions such as heart pump function decline and aging, heart disease, heart failure, myocardial infarction, stroke, etc. The blood pressure recognition unit (1420) can derive highly accurate predictive results regarding the subject's health status by repeatedly measuring and using reliable pulse pressure information.

Furthermore, the Blood pressure recognition unit (1420) can recognize the subject's blood pressure values, including the sensing mean blood pressure value (sMBP), sensing pulse pressure value (sPP), sensing systolic blood pressure value (sSBP), and sensing diastolic blood pressure value (sDBP).

The information provision unit (1430) provides measurement results that sequentially include the systolic and diastolic blood pressure values recognized by the blood pressure recognition unit (1420) while repeating the inflation and deflation of the cuff for the set number of cycles. At this time, the information provision unit (1430) can provide multiple systolic and diastolic blood pressure measurements collectively after the set number of repeated measurements is completed.

In addition, the blood pressure measuring device (10), according to one embodiment, may further include a voice support function that outputs a voice message indicating that an abnormal blood pressure measurement value has been detected.

In one embodiment, the information provision unit (1430) can provide the measurement results in paper form by interfacing with a thermal printer or other small printers or through an input/output interface (130) such as an LCD or LED display screen provided in the automatic repetitive blood pressure measurement of a smart blood pressure monitor (10). In this case, the display screen can provide the measurement results in real-time, displaying multiple systolic and diastolic blood pressure measurement values sequentially on one screen as the measurements progress.

In one embodiment, the information provision unit (1430) further provides the measurement results, which sequentially include the repeatedly measured systolic and diastolic blood pressure values, to the manager terminal (25) and subject terminal (20). The information provision unit (1430) can provide this information to a service server (40) through a network (30). Then, the service server (40) can recognize the information of the subject whose blood pressure is being measured through the automatic repetitive blood pressure measurement of a smart blood pressure monitor (10) and deliver the measurement results to the manager terminal (25) held by the manager of the blood pressure measuring device (10) and the subject terminal (20) held by the subject.

To this end, the automatic repetitive blood pressure measurement of a smart blood pressure monitor (10), according to one embodiment, can further receive the subject information by entering a patient code or recognizing a barcode.

Additionally, the service server (40) can be implemented to collect and manage the blood pressure measurement results for each subject by aggregating the measurement results from the automatic repetitive blood pressure measurement of a smart blood pressure monitor (10) according to one embodiment.

Furthermore, the information provision unit (1430) can calculate and provide the average values of the recognized systolic and diastolic blood pressure values during the repeated inflation and deflation of the cuff for the set number of cycles.

By providing the average values of multiple systolic blood pressure measurements and diastolic blood pressure measurements measured for each of the multiple cycles, along with the respective systolic blood pressure measurements and diastolic blood pressure measurements, an overall measurement value can be obtained.

In an additional embodiment of the present invention, the re-measurement request unit (1440) outputs a re-measurement request message through an input/output interface (130) such as an LCD or LED display screen if the difference between the recognized systolic and diastolic blood pressure values exceeds a set threshold during the repeated inflation and deflation of the cuff for the set number of cycles.

The blood pressure measuring device (10), according to one embodiment, can request additional measurements if the accuracy of the recognized systolic and diastolic blood pressure values is recognized to be below the standard, even after repeating the blood pressure measurements for the set number of cycles. In this case, if the accuracy is below the standard, additional automatic measurements can be taken even without the subject's approval.

The re-measurement request unit (1440) can remove the blood pressure measurements of the cycles where the recognized systolic and diastolic blood pressure values have deviations exceeding the standard and include the additional measured data to be used as blood pressure measurement and analysis data. In other words, it can be implemented to derive blood pressure measurement results by selecting high-accuracy data.

Therefore, the blood pressure measuring device (10), according to one embodiment, not only performs multiple blood pressure measurements with a single operation signal input, but also automatically excludes the detected abnormal measurements and re-measures the blood pressure values if it is recognized that there are errors in the measurements due to sudden movements, etc., thereby improving accuracy.

In this case, the blood pressure measuring device (10), according to one embodiment, may further include a voice support function that outputs a voice message indicating that an abnormality in the blood pressure measurement value has been detected. For example, messages like, “An abnormality is suspected in the measurement value,” or “Would you like to perform a re-measurement?” can be output in voice form to notify the user of the detected abnormality and prompt them to perform a re-measurement for accurate measurement.

In one embodiment, the blood pressure measuring device (10) can repeatedly measure blood pressure for a set number of cycles and calculate basic statistics such as the average value and standard deviation. Based on the standard deviation, values outside a certain range of standard deviation can generally be considered outliers. The blood pressure manager can set the range of standard deviation for identifying outliers.

However, this is not limited to this method alone. It can also implement the IQR removal technique, which uses the interquartile range representing the middle 50% of the data to identify and remove outliers to distinguish whether the measured values are abnormal.

Thus, with a single operation signal input, it can derive multiple systolic and diastolic blood pressure measurements, exclude the values identified as outliers from the derived multiple systolic and diastolic blood pressure measurements, and accept additional measurements to improve accuracy. The range of standard deviation for determining outliers can be set directly and modified by the blood pressure manager.

In another embodiment, the automatic repetitive blood pressure measurement of a smart blood pressure monitor (10) provides additional information about the remaining measurement cycles required to reach the set number of cycles during the repeated inflation and deflation of the cuff when a single operation signal is input.

In one embodiment, when a single operation signal is an input and the measurement is set to three cycles, the device can notify the subject and medical staff, such as blood pressure managers, in real-time through a display screen such as an LED or LCD on the device about how many measurement cycles remain as the cuff inflates and deflates.

For example, if the measurement is set to repeat three times, messages such as “2 out of 3 measurements remaining,” “1 out of 3 measurements remaining,” can be displayed, allowing the subject to roughly understand how long they need to maintain their measurement posture until the measurement is complete. This feature provides convenience by allowing the subject to know the measurement results after each cycle is completed and stop the measurement before reaching the set number of cycles if desired.

In one embodiment, the automatic repetitive blood pressure measurement of a smart blood pressure monitor (10) is formed as part of a band that attaches to a portion of the subject's body. The automatic repetitive blood pressure measurement of a smart blood pressure monitor (10) can be implemented, for example, in the form of a bracelet that attaches to the wrist. The subject can measure blood pressure while lying down.

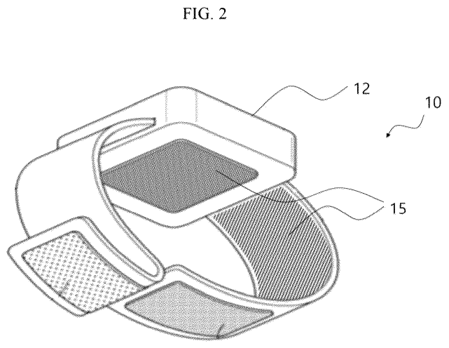

FIG. 2 is an illustration of the appearance of a wrist-worn automatic repetitive blood pressure measurement of a smart blood pressure monitor according to one embodiment.

In one embodiment, the automatic repetitive blood pressure measurement of a smart blood pressure monitor (10) includes an operation interface for starting blood pressure measurement.

When a single operation signal is generated through the operation interface, the device performs blood pressure measurement operations three times consecutively with a band-type blood pressure measurement unit, accumulates the blood pressure measurement results, and then outputs the results for each cycle after the measurement is completed, which can be implemented in a form that includes a blood pressure measurement information display unit. The operation interface and the blood pressure measurement information display unit can be physically implemented as a single configuration (12). The blood pressure measurement unit can be implemented with a cuff (15) mounted on a bracelet-shaped band that can be attached to a part of the wrist.

In one embodiment, the automatic repetitive blood pressure measurement of a smart blood pressure monitor (10) is implemented as a wrist-worn type, as shown in FIG. 2, providing portability and convenience. That is, the wrist-worn type is small and lightweight, making it easy to carry and convenient to use while traveling or going out. Additionally, the wrist-worn type only needs to be worn on the wrist, which makes the wearing process relatively simpler and more convenient compared to the upper arm blood pressure monitor type.

Moreover, the wrist-worn type has higher accessibility for elderly individuals or subjects with limited mobility who find it difficult to lift their arms high. It can be worn easily with one hand, making it convenient for independent blood pressure measurement. Its small size and lightweight design allow for blood pressure measurement without being restricted by time or place. Specifically, it is implemented in a way that can quickly and accurately measure the subject's blood pressure without pairing it with the cuffs of a wrist-type smartwatch. The measurement results can be provided to the subject terminal (20).

In an additional embodiment, the information provision unit (1430) of the automatic repetitive blood pressure measurement of a smart blood pressure monitor (10), according to one embodiment, provides further information on the temporal changes in the measurement results and recommended diet information based on the calculated average values of systolic and diastolic blood pressure over a certain period.

The information provision unit (1430) can graphically present the temporal changes in the average blood pressure values over a certain period based on the measured average blood pressure values through the linked subject terminal (20). The service server (40) can be implemented to accumulate and manage the measurement results of the subject.

Additionally, if the average blood pressure value of the subject deviates from the normal range, it can provide recommended diet adjustment information necessary to normalize the average blood pressure value and further implement a diet adjustment function. Moreover, it can also provide exercise information necessary for blood pressure control. Furthermore, it can be implemented to share the measured blood pressure information in conjunction with diet adjustment apps or health and exercise-related apps running on the subject terminal (20).

In this case, the diet and exercise-related information necessary for blood pressure control can be implemented with information collected by the service server (40) from medical institutions or experts. However, this is not limited to this alone, and it can also be based on data directly input and managed by the blood pressure measurement manager through the manager terminal (25).

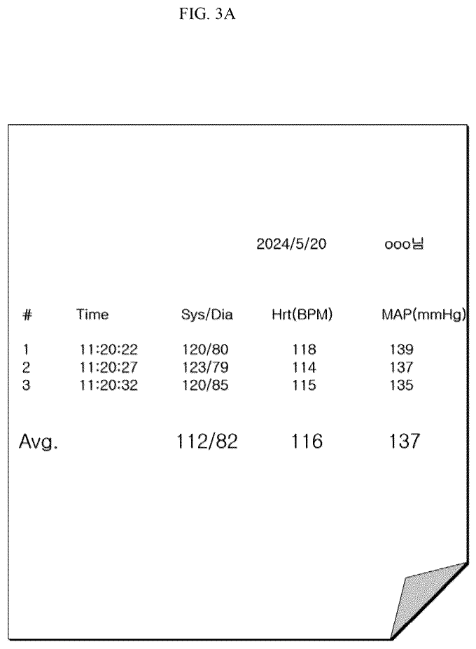

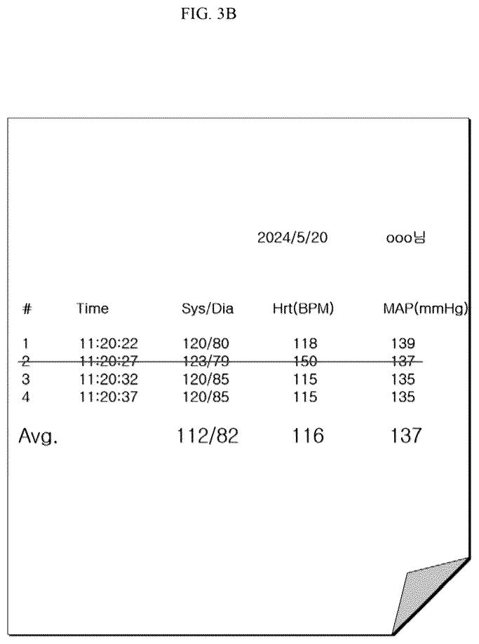

FIGS. 3A and 3B are illustrations to explain the form in which measurement results are provided from the information provision unit in an embodiment.

In one embodiment, the information provision unit (1430) can automatically measure three times for one measurement operation signal input, as shown in FIG. 3A, and provide the measurement results on a single screen or printed on a single page. In this case, the automatic repeated measurement cycle can be set by the blood pressure manager.

The information provision unit (1430) excludes any anomalous measurement values detected in the repeated blood pressure measurement results and provides additional measurements. In this case, as shown in FIG. 3B, the excluded anomalous measurements can be visibly identified.

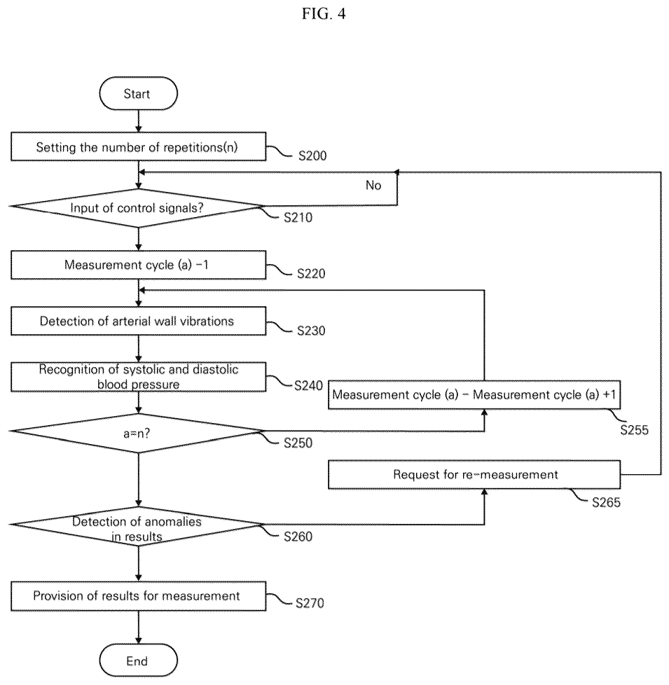

FIG. 4 is a flowchart illustrating an automatic repetitive blood pressure measurement method of a smart blood pressure monitor according to an embodiment of the present invention.

In one embodiment, the automatic repetitive blood pressure measurement method of the smart blood pressure monitor first receives a single operation signal from the blood pressure measurement manager to set the number of repetitions (n) for inflating and deflating the cuff (S200).

The number of repetitions (n) to be measured can be input through the input/output interface provided in the automatic repetitive blood pressure measurement of a smart blood pressure monitor or set through the network by the manager terminal. For example, it can be set to measure 3 or 4 times repeatedly.

Then, upon receiving a single operation signal (S210), the cuff is periodically inflates and deflates for the preset number of cycles (S220). During the inflation and deflation of the cuff, the pressure in the cuff is between the systolic and diastolic pressures of the artery, and the vibrations of the arterial wall are detected (S230).

Based on the detected vibrations of the arterial wall, the point at which the maximum vibration is detected is recognized as the systolic blood pressure (S240), and if the amount of vibration decrease exceeds a threshold, it is recognized as the diastolic blood pressure (S240).

However, they are not limited to these steps. Upon receiving a single operation signal, the cuff inflates and deflates for the set number of repetitions, and the pulse pressure value is further detected to assess the heart's pumping function, aging state, heart disease, heart failure, myocardial infarction, stroke, and other risk factors.

Additionally, the subject's blood pressure values, including sensing mean blood pressure (sMBP), sensing pulse pressure (sPP), sensing systolic blood pressure (sSBP), and sensing diastolic blood pressure (sDBP) can be recognized.

Then, the measurement cycle (a) for systolic and diastolic blood pressure is incremented by 1 and repeated until the measurement cycle (a) reaches the preset number of repetitions (n) (S250, S255). In one embodiment, if the difference between the recognized systolic and diastolic blood pressure values during the repetition of inflation and deflation of the cuff for the set number of cycles (n) exceeds a threshold (S260), a re-measurement request message is output (S265).

Although blood pressure is repeatedly measured for the set number of repetitions (n), additional measurements can be requested if the recognized systolic and diastolic blood pressure accuracy is considered below the standard. If the accuracy is below the threshold, additional automatic measurements can be performed without the subject's approval.

Moreover, if the deviation between the recognized systolic blood pressure measurement value and the diastolic blood pressure measurement value exceeds the threshold, the blood pressure measurement values of that cycle can be excluded, and additional measured data can be included to be used as blood pressure measurement and analysis data. In other words, the system can select and derive blood pressure measurement results from high-accuracy data.

Therefore, the blood pressure measurement method, according to one embodiment, not only performs multiple blood pressure measurements with a single operation signal input, but also automatically excludes anomalous measurements in case of detected errors due to sudden movements, etc., and performs additional re-measurements to improve accuracy.

During the repetition of the cuff inflation and deflation for the set number of cycles, the systolic and diastolic blood pressure measurement values recognized in the recognition step are provided sequentially as measurement results (S270).

In one embodiment, the step of providing measurement results further provides the measurement results, including the systolic and diastolic blood pressure values in sequence to the manager terminal and subject terminal.

Additionally, the step of providing measurement results includes calculating and providing the average values of the recognized systolic and diastolic blood pressure values during the repetition of the cuff inflation and deflation for the set number of cycles.

Moreover, upon receiving a single operation signal, while repeating the cuff inflation and deflation for the set number of cycles, the system can provide additional information on the remaining measurement cycles needed to complete the set number of cycles. For example, if the set number of cycles is three, the system can output messages such as “2 out of 3 measurements remaining,” “1 out of 3 measurements remaining,” allowing the subject to know approximately how long they need to maintain the measurement posture until completion. This also provides the convenience of allowing the subject to stop the measurement after each cycle if they wish to know the results before completing the set number of cycles.

The aforementioned method can be implemented as an application or in the form of program instructions that can be executed through various computer components and recorded on a computer-readable recording medium. The computer-readable recording medium can include program instructions, data files, data structures, or combinations thereof.

The program instructions recorded on the computer-readable recording medium can be specially designed and configured for the present invention or can be publicly known and available to those skilled in the field of computer software.

Examples of computer-readable recording media include magnetic media such as hard disks, floppy disks, and magnetic tapes; optical recording media such as CD-ROMs and DVDs; magneto-optical media such as floptical disks; and hardware devices specially configured to store and execute program instructions, such as ROM, RAM, and flash memory.

Examples of program instructions include not only machine code generated by a compiler, but also high-level language code that can be executed by a computer using an interpreter, etc. The hardware device can be configured to operate as one or more software modules to perform processing according to the present invention, and vice versa.

Although the above has been described with reference to embodiments, those skilled in the art will understand that the present invention can be variously modified and changed without departing from the spirit and scope of the invention as defined in the following claims.

[Description of Symbols]

| | |
|---|---|
| 10: Device for Automatic Repetitive | |
| Blood Pressure Measurement | |
| 20: Subject Terminal | |
| 25: Manager Terminal | 110: Communication Interface |
| 120: Memory | 130: Input/output Interface |
| 140: Processor | 1410: Detection Unit |
| 1420: Blood Pressure Recognition | 1430: Information Provision |
| Unit | Unit |
| 1440: Re-measurement Request Unit | |

What is claimed is:

1. A method for automatic repetitive blood pressure measurement of a smart blood pressure monitor, performed in a computing device comprising one or more processors and memory storing one or more programs executed by the one or more processors, the method comprising the following steps: Periodically repeating the inflation and deflation of a cuff for a predetermined number of cycles upon receiving a single operation signal input, detecting arterial wall vibrations when the cuff pressure is between the systolic and diastolic pressures during the inflation and deflation of the cuff; Recognizing the point of maximum vibration as the systolic blood pressure based on the detection of arterial wall vibrations, and recognizing the diastolic blood pressure if the decrease in vibrations exceeds a threshold value; and providing measurement results that sequentially include the recognized systolic and diastolic blood pressure values during the repeated inflation and deflation of the cuff for the predetermined number of cycles.

2. The method of claim 1, further comprising providing measurement results, sequentially including systolic and diastolic blood pressure values, to a manager terminal and a subject terminal.

3. The method of claim 1, wherein the step of providing the measurement result refers to the automatic repetitive blood pressure measurement method of the smart blood pressure monitor, which calculates and provides the average value of the systolic and diastolic blood pressure measurements recognized during the recognition step while inflating and deflating the cuff for a set number of cycles.

4. The method of claim 3, further comprising the step of outputting a remeasurement request message if the difference between the systolic and diastolic blood pressure values recognized in the recognition step exceeds a set threshold while repeating the inflation and deflation of the cuff for a specified number of cycles.

5. The method of claim 1, further comprising, upon receiving a single operation signal input, repeating the inflation and deflation of the cuff for a set number of cycles and additionally providing information on the remaining measurement cycles required to reach the set number of cycles for inflation and deflation of the cuff.

6. A device for automatic repetitive blood pressure measurement of a smart blood pressure monitor is a computer device equipped with one or more processors and memory that stores one or more programs executed by the one or more processors, which includes the following features: Upon receiving a single operation signal, the cuff inflates and deflates periodically for a set number of cycles; During inflation and deflation, the cuff pressure is between the systolic and diastolic pressures of the artery, and a detection unit detects the vibrations of the arterial wall; Based on the detection results from the detection unit, the point at which maximum vibration is detected is recognized as the systolic blood pressure, and if the vibration decreases beyond a certain threshold, it is recognized as the diastolic blood pressure by the blood pressure recognition unit; While the cuff inflates and deflates for the set number of cycles, the information provision unit provides the measurement results, which include the recognized systolic and diastolic blood pressure values in sequence.

7. The device of claim 6, wherein the information provision unit is a automatic repetitive blood pressure measurement of a smart blood pressure monitor that further provides the measurement results, including the systolic and diastolic blood pressure values sequentially, to the manager terminal and subject terminal.

8. The device of claim 6, wherein the information provision unit is a automatic repetitive blood pressure measurement of a smart blood pressure monitor that calculates and provides the average value of the systolic and diastolic blood pressure values recognized during the step of repeating the inflation and deflation of the cuff for the set number of cycles.

9. The device of claim 8, further comprising a re-measurement request unit, which outputs a re-measurement request message if the difference between the recognized systolic and diastolic blood pressure values exceeds a certain threshold during the step of repeating the inflation and deflation of the cuff for the set number of cycles.

10. The device of claim 6, wherein the device additionally provides the remaining measurement cycle information required to reach the set number of inflation and deflation cycles of the cuff while repeating the inflation and deflation for the set number of cycles upon receiving a single operation signal.

11. The device claim 6, wherein the device means a automatic repetitive blood pressure measurement of a smart blood pressure monitor, where the cuff is formed as part of a band that is attached to a portion of the subject's wrist.

12. The device of claim 8, wherein the information provision unit of the automatic repetitive blood pressure measurement of a smart blood pressure monitor provides additional information on the temporal changes in the measurement results of the subject and recommended dietary information based on the average of the calculated systolic and diastolic blood pressure values over a certain period.
